(19) United States
(12) Patent Application Publication
(10) Pub. No.: US 2025/0169675 A1
(43) Pub. Date: May 29, 2025

(54) PHOTO OR IMAGE ASSISTED MEDICAL DEVICES FOR DELIVERING ENERGY AND/OR FLUID TO TISSUE AND RELATED METHODS

(71) Applicant: Boston Scientific Scimed, Inc., Maple Grove, MN (US)

(72) Inventors: Austin Grant JOHNSON, Worcester, MA (US); George Wilfred DUVAL, Sudbury, MA (US); Allison KUMARASENA, Wellesley, MA (US)

(73) Assignee: Boston Scientific Scimed, Inc., Maple Grove, MN (US)

(21) Appl. No.: 18/960,161

(22) Filed: Nov. 26, 2024

Related U.S. Application Data
(60) Provisional application No. 63/603,204, filed on Nov. 28, 2023.

(51) Int. Cl. A61B 1/00; A61B 1/015; A61B 1/018; A61B 1/07; A61B 17/00; A61B 18/00; A61B 18/14

(52) U.S. Cl. CPC A61B 1/00006; A61B 1/00009; A61B 1/00066; A61B 1/00126; A61B 1/015; A61B 1/018; A61B 1/07; A61B 18/14; A61B 2017/00061; A61B 2018/00642; A61B 2018/00708; A61B 2018/00982; A61B 2018/1405

(57) ABSTRACT

A medical device includes an insertion portion, a light-emitting optical fiber, and a light-receiving optical fiber. The insertion portion includes a shaft, and the shaft includes a sheath and an electrode. The sheath includes a sheath lumen, and the electrode is positioned within at least a distal portion of the sheath. The electrode includes an electrode lumen, and the electrode is configured to deliver energy to a target site. The light-emitting optical fiber is configured to deliver light to the target site. At least a portion of the light-emitting optical fiber is positioned within the sheath or the electrode lumen. The light-receiving optical fiber is configured to receive light from the target site. At least a portion of the light-receiving optical fiber is positioned within the electrode lumen.

### CROSS-REFERENCE TO RELATED APPLICATIONS

This application claims the benefit of priority of U.S. Provisional Patent Application No. 63/603,204, filed on Nov. 28, 2023, the entirety of which is incorporated herein by reference.

### TECHNICAL FIELD

Aspects of this disclosure generally relate to photo or image assisted medical devices for delivering energy or fluid to tissue and related methods. Embodiments of the disclosure relate to medical devices and related methods for the treatment of tissue by delivering electrical energy to or into tissue and/or injecting fluid into and/or under tissue.

### BACKGROUND

Medical devices, such as endoscopes or other suitable insertion devices, are employed for various diagnostic and surgical procedures, such as endoscopy, laparoscopy, arthroscopy, etc. Many of these procedures involve delivering energy to tissue, delivering fluid to the tissue, and/or cutting the tissue to treat tumors, infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy. Such procedures may be carried out by inserting an insertion device into a subject's body through a surgical incision or via a natural anatomical orifice and performing a procedure or operation that may involve cutting at a target site.

In some aspects, a user may use visual cues to determine what anatomy to cut, inject into, or otherwise treat. The user may visually determine that the color of the tissue is red and is likely a blood vessel, so cutting should not take place. On the other hand, if the color of the tissue is white or blue, then submucosal tissue may be present, and cutting, injection, or other treatment should be performed. In some aspects, the user may spend a substantial amount of time and/or effort determining or otherwise assessing tissue color in the submucosal tissue layers. Additionally, in some aspects, the user may err in the assessment, and thus cut, inject into, or otherwise treat tissue that is not in need of cutting or treatment, which may then require cauterization or other hemostatic treatments. These additional steps may increase the costs, duration, and/or risks of the procedure. Therefore, there is a need for a device or method that may assist the user in distinguishing between tissue types or locations, for example, to help decrease the costs, duration, and/or risks of a procedure.

The devices and methods of this disclosure may rectify one or more of the deficiencies described above or address other aspects of the art.

### SUMMARY

Examples of the disclosure relate to, among other things, medical devices with optical photo or imaging assistance for distinguishing between tissue types, delivering electrical energy or otherwise treating tissue, and delivering fluid into and/or under the tissue. Each of the examples disclosed herein may include one or more of the features described in connection with any other disclosed examples.

In an example, a medical device may comprise: an insertion portion, which may include a shaft. The shaft may include a sheath and an electrode. The sheath may include a sheath lumen, and the electrode may be positioned within at least a distal portion of the sheath. The electrode may include an electrode lumen, and may be configured to deliver energy to a target site. The medical device may also include a light-emitting optical fiber, which may be configured to deliver light to the target site. At least a portion of the light-emitting optical fiber may be positioned within the sheath or the electrode lumen. The medical device may also include a light-receiving optical fiber, which may be configured to receive light from the target site. At least a portion of the light-receiving optical fiber may be positioned within the electrode lumen.

Any examples described herein may have any of these features alone or in combination. The electrode may have a “T” shaped cross-section with an electrode shaft and a widened distal portion. A portion of the electrode may extend distally of the sheath lumen, and the widened distal portion may be wider than an opening of the sheath lumen. A portion of the sheath may be configured to convey light and form the light-emitting optical fiber. A distal end of the sheath may include a first width, and a distal end of the electrode may include a second width. The second width may be less than the first width. A width of the electrode lumen may be greater than a width of the light-receiving optical fiber, such that a portion of the electrode lumen may form a fluid delivery portion that may occupy a space adjacent to the light-receiving optical fiber. The light-emitting optical fiber may be positioned within the electrode lumen. The sheath may include one or more fluid delivery or suction application lumens. The one or more fluid delivery or suction application lumens may include at least two fluid delivery or suction application lumens. The respective distal openings of the one or more fluid delivery or suction application lumens may be at least partially radially outside of a distal end of the electrode. The sheath may include the light-emitting optical fiber and the one or more fluid delivery or suction application lumens. The medical device may include a handle, and the light-emitting optical fiber may extend from the handle through a portion of the shaft to deliver light to the target site. The handle of the medical device may include a port, and the port may be coupled to a light source that provides light to the light-emitting optical fiber. The light-receiving optical fiber may extend through the electrode lumen, through a shaft lumen, to the handle, and to a processor, where the processor may analyze light received by the light-receiving optical fiber to generate a light profile. Based on the generated light profile, the processor may be configured to deactivate the electrode. The processor may detect a red light, and the processor may send a signal to disable the energization of the electrode.

In another example, a medical device may comprise a handle including a port, a hub, and an insertion portion. The insertion portion may include a shaft, and the shaft may include a sheath and an electrode. The sheath may include a sheath lumen, and the electrode may be positioned within at least a distal portion of the sheath. The electrode may include an electrode lumen, and the electrode may be configured to deliver energy to a target site. The light-emitting optical fiber may be configured to deliver light to the target site, and at least a portion of the light-emitting optical fiber may be positioned within the sheath or the electrode lumen. A light-receiving optical fiber may be configured to receive light from the target site. At least a portion of the light-receiving optical fiber may be positioned within the electrode lumen, and at least a portion of the sheath may direct optical energy distally.

Any of the devices disclosed herein may include any of the following features in any combination. The sheath may be configured to emit light, and the sheath may emit light toward the target site. At least a portion of the sheath may be configured to convey light and form the light-emitting optical fiber. A width of the electrode lumen may be greater than a width of the light-receiving optical fiber, such that a portion of the electrode lumen may form a fluid delivery portion that occupies a space adjacent to the light receiving optical fiber. The light-receiving optical fiber may be coupled to a processor. The processor may analyze light received by the light-receiving optical fiber to generate a light profile, and, based on the generated light profile, the processor may be configured to deactivate the electrode. If the processor detects a red light, the processor may send a signal to disable the energization of the electrode.

In another example, a medical device may comprise an insertion portion, including a sheath and an electrode. The sheath may include a sheath lumen. The sheath may include one or more fluid delivery or suction application lumens. The electrode may be positioned within at least a distal portion of the sheath lumen, and the electrode may include an electrode lumen. The electrode may be configured to deliver energy to a target site. A light-emitting optical fiber may be configured to deliver light to the target site, and at least a portion of the light-emitting optical fiber may be positioned within the sheath or the electrode lumen. A light-receiving optical fiber may be configured to receive light from the target site, and at least a portion of the light-receiving optical fiber may be positioned within the electrode lumen. The light-receiving optical fiber may be coupled to a processor, and the processor may analyze light received by the light-receiving optical fiber to generate a light profile. Based on the generated light profile, the processor may be configured to deactivate the electrode. The processor may detect a red light, and the processor may send a signal to disable the energization of the electrode.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and, together with the description, explain the principles of the disclosure.

FIG. 1 illustrates a side view of an exemplary medical device and an enlarged cross-sectional view of a distal portion of the exemplary medical device.

FIG. 2 illustrates a cross-sectional view of a distal portion of another exemplary medical device.

FIG. 3 illustrates a cross-sectional view of a distal portion of an alternative exemplary medical device.

FIG. 4 illustrates a cross-sectional view of a distal portion of another exemplary medical device.

FIG. 5 illustrates a cross-sectional view of a distal portion of yet another exemplary medical device.

### DETAILED DESCRIPTION

Examples of the disclosure include devices and methods for facilitating and improving the efficacy, efficiency, and safety of treating or manipulating tissue when, for example, applying electrical energy to tissue with an electrode, delivering fluid into and/or under tissue during a medical procedure through the distal end of the electrode.

In one or more aspects of the disclosure, the medical device may allow a user (e.g., physician, medical technician, or other medical service provider) to deliver and receive or detect light while the device cuts, dissects, coagulates, cauterizes, or otherwise treats tissue. The light received may be transmitted to capital equipment to process the characteristics of the incoming light, for example, to help determine whether the medical device should be enabled or deactivated. For example, if the incoming light has a light profile that characterizes a blood vessel, then the medical device may emit a warning, may be turned off, may be deactivated, etc. to help avoid damage to the blood vessel. However, if the incoming light characterizes submucosal tissue, the medical device may remain active or enabled, for example, energized to help cut the submucosal tissue. In any of these aspects, the user may also manually control (e.g., activate or deactivate) the medical device.

Further aspects of the disclosure may provide a user with the ability to apply electrical energy or heat to tissue using a medical device having an electrode. In some embodiments, aspects of the disclosure may provide the user with the option of delivering fluid into or under tissue with the same medical device. In these aspects, the medical device may combine light-receiving and emitting components in the electrode with an optional fluid delivery component.

Reference will now be made in detail to examples of the disclosure described above and illustrated in the accompanying drawings. The same reference numbers will be used throughout the drawings to refer to the same or like parts wherever possible.

The terms “proximal” and “distal” are used herein to refer to the relative positions of the components of an exemplary medical device. When used herein, “proximal” refers to a position relatively closer to the exterior of a subject's body or closer to a user, such as a medical professional holding or otherwise using the medical device. In contrast, “distal” refers to a position relatively further away from the medical professional or other user holding or otherwise using the medical device or closer to the interior of the subject's body. As used herein, the terms “comprises,” “comprising,” “having,” “including,” or other variations thereof are intended to cover a non-exclusive inclusion, such that a device or method that comprises a list of elements does not include only those elements but may include other elements not expressly listed or inherent thereto. Unless stated otherwise, the term “exemplary” is used in the sense of “example” rather than “ideal.” As used herein, the terms “about,” “substantially,” and “approximately” indicate a range of values within +/−10% of a stated value.

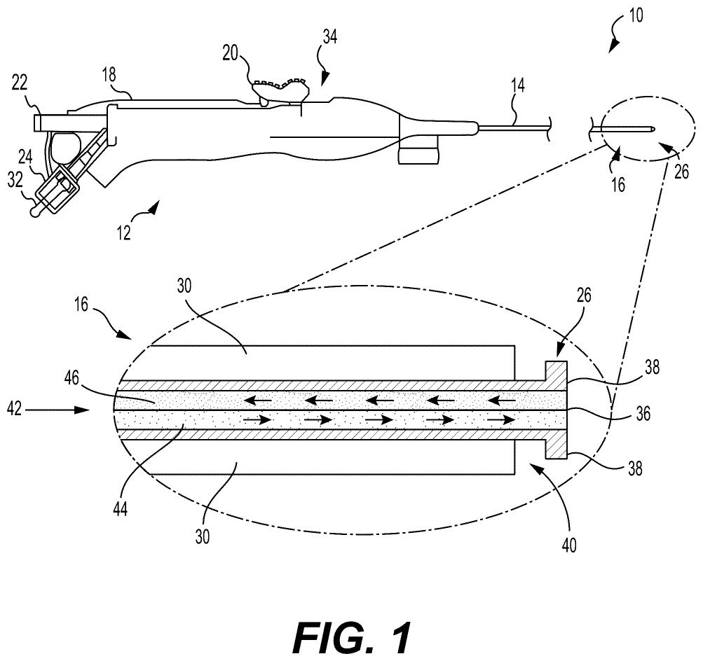

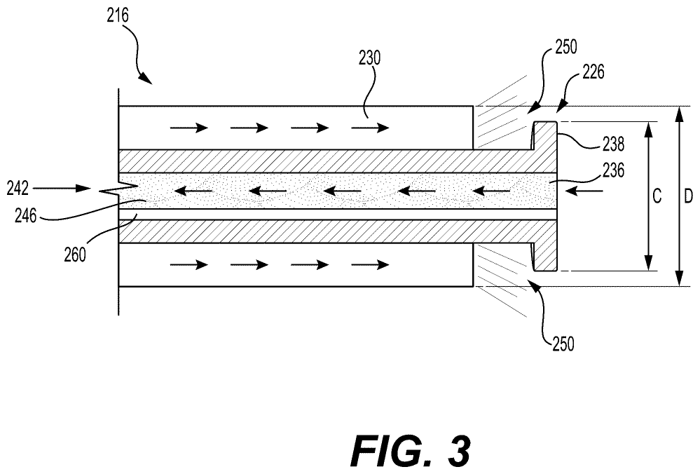

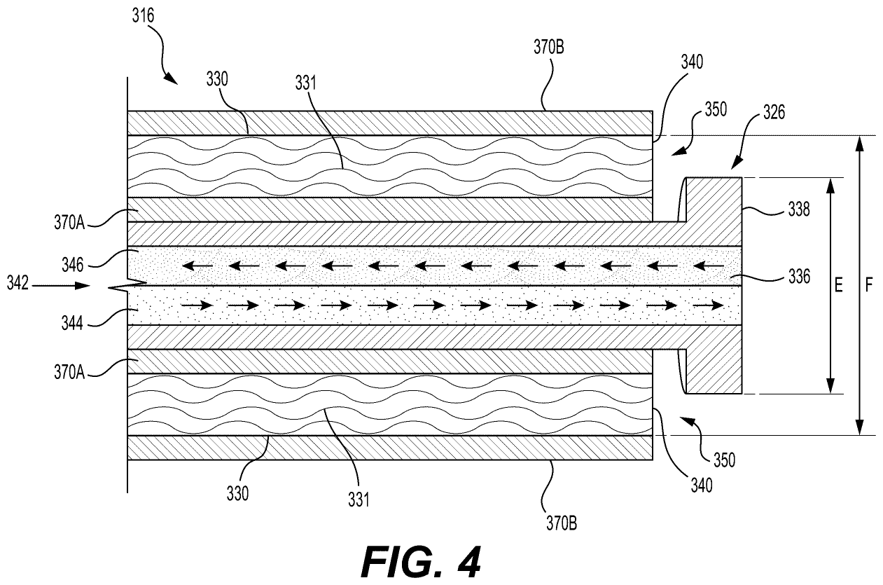

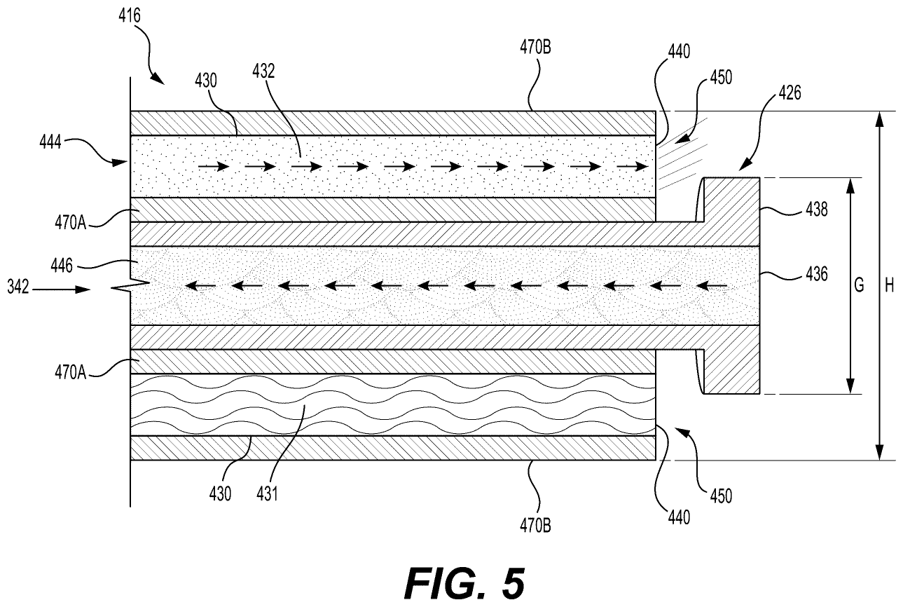

FIG. 1 depicts a medical device 10, including a handle 12, a shaft 14, and a distal end 16. The handle 12 may include a main body 18 and a movable body 20. The handle 12 may include a port 22 configured to receive optical or light energy, and the handle 12 may also include a hub 24 configured to receive electrical energy from an electrical plug or socket. The handle 12 may additionally include a fluid port (not shown) that may be coupled to a fluid source connected to a fluid delivery portion (FIG. 3), the fluid port may be configured to provide a fluid to a sheath in FIG. 4, or at least a lumen of the sheath in FIG. 5. Alternatively, the port 22 may be configured to receive both optical energy and/or fluid. The distal end 16 may include a portion of the shaft 14. The shaft 14 may include a sheath 30. The sheath 30 may include an inner lumen 42 that radially surrounds an electrode 26. The distal end 16 may include an end effector, for example, the electrode portion 26 (hereinafter “electrode 26”). The electrode 26 may be electrically connected to the hub 24. Additionally, the electrode 26 may be optically connected to the port 22. In some aspects, as discussed below, the electrode 26 may include an insulation tip (not shown), which may at least partially surround a distal portion of the electrode 26.

The port 22 may be positioned on a proximal portion of the main body 18, for example, a proximal end of the main body 18. Alternatively, the port 22 may be positioned on a distal or central portion of the main body 18. The handle 12 may be coupled to capital equipment (not shown) by the port 22. The port 22 may receive light or optical energy from capital equipment, including a light source transmitted to the port 22. The port 22 may be in optical communication with the electrode 26 via the sheath 30 with an internal lumen (not shown), which may extend through the handle 12 and the shaft 14. The internal lumen may extend longitudinally through the main body 18 of handle 12 and shaft 14 to connect the port 22 to electrode 26. As discussed in more detail below, the electrode 26 or, in some aspects, the sheath 30 may include an internal lumen configured to direct, emit, or otherwise receive light or a flow of optical energy.

As discussed in more detail below, after the port 22 receives light or optical energy from the capital equipment, the components at the distal end 16 (e.g., one or more optical fibers 44, 46) may emit light onto or toward the target site. Light, imaging, or light energy may also be received by, for example, the optical fibers 44, 46 at the distal end 16. The light or imaging received may travel through an internal lumen of the sheath 30 and be received by capital equipment with sensors or photo-diodes that detect, analyze, or otherwise react to the characteristics of the received light. The capital equipment may have a processor, a central processing unit (CPU) or a microcontroller responsible for analyzing data received from embodiments, including optical properties (e.g., optical fibers 44 and 46, sheath 130). The processor may run or perform one or more algorithms that interpret the optical signals and emit one or more signals based on predefined criteria.

The hub 24 may be positioned on the main body 18, for example, on the proximal end of the main body 18. The handle 12 may also be coupled to an energy source (not shown) through hub 24. The hub 24 may include one or more prongs or pins 32 to couple to the energy source. The hub 24 may also be electrically coupled to the electrode 26 via a conductive element (not shown), which may be electrically coupled to the pin 32 and extend through the handle 12 and at least a portion of the shaft 14. The energy source may be an electrocautery source, a radio frequency generator, a heating source, a current generator, etc.

In another aspect, the pin 32 may extend through the hub 24 transverse to a longitudinal axis of the handle 12. The pin 32 may be electrically and physically connected to the conductive element, which may be a wire, a cable, or a braided sheath. The conductive element may be electrically conductive or include an electrically conductive element and may extend longitudinally through an internal lumen and the shaft 14. In another aspect, fluid delivered through an optional fluid port (not shown) may surround at least a portion of the conductive element. In one aspect, the conductive element may include one or more insulation layers to help insulate the conductive element from the fluid in the internal lumen. Although not shown, in another aspect, the energy source may be a part of or coupled to the handle 12 (e.g., an internal battery in the handle 12) and act as the sole energy source or as a supplement to the hub 24 coupling to an external energy source.

In another aspect, the port 22 may extend from the proximal end of the main body 18 in a direction parallel to a longitudinal axis of the main body 18. The hub 24 may extend from the proximal end of the main body 18 at an angle transverse (e.g., approximately 45 degrees) to the longitudinal axis of main body 18. In another aspect, the hub 24 may be positioned on a distal or central portion of the main body 18 or the movable body 20. Although not shown, the main body 18 and the hub 24 may include a one-way valve, a seal, threading, or any appropriate element to help maintain a secure connection between the handle 12 and the energy source, minimize or prevent back-flow (e.g., fluid flowing from an optional fluid port or internal lumen and proximally out of the hub 24).

The medical device 10 may be inserted into the body lumen of a subject, either through an insertion device (not shown) or alone, such that at least a portion of the shaft 14 may be within the subject. At the same time, the handle 12 may remain outside of the subject. The distal end 16 may be positioned at or adjacent to a target site within the subject. From outside of the subject, a user can manipulate the handle 12. The movement of the movable body 20 relative to the main body 18 in a first direction (e.g., the distal direction) may extend the electrode 26 relative to the sheath 30 (e.g., positions electrode 26 distally relative to a distal end of the shaft 14), while movement of the movable body 20 relative to the main body 18 in a second direction (e.g., the proximal direction) may retract the electrode 26 relative to the sheath 30 (e.g., positions the electrode 26 proximally relative to a distal end of the sheath 30). Although not shown, the movable body 20 or additional components of the handle 12 may articulate the electrode 26 (or the electrode 26 and the distal end 16) left or right and/or up or down relative to the shaft 14.

The movable body 20 may be coupled to a drive element, and the drive element may impart the distal or proximal movement to at least a portion of the electrode 26 based on the relative movement between the main body 18 and the movable body 20. In one aspect, the conductive element may also act as a drive wire, rod, cable, or the like, such that the conductive element imparts distal or proximal movement to at least a portion of the electrode 26 while also coupling the electrode 26 to the hub 24, (e.g., the one or more pins 32,) to deliver the electrical energy to (and/or from) the electrode 26. The movable body 20 may be coupled to the conductive element via a coupling mechanism, for example, a coupler (not shown). In one aspect, the coupler may be physically coupled (either directly or indirectly) to movable body 20 and may also be physically coupled (either directly or indirectly) to the conductive element such that movement of movable body 20 extends or retracts the conductive element, and thus extends or retracts electrode 26. It is noted that the coupler and/or other components within handle 12 may help maintain the electrical connection between pin 32 and the conductive element when the conductive element, and thus the electrode 26, is in the retracted or extended positions.

FIG. 1 includes an enlarged cross-sectional view of the distal end 16 and the electrode 26. The distal end 16 is at a distal portion of the shaft 14 of the medical device 10. As shown in FIG. 1, the shaft 14 extends from a distal portion of the main body 18 to the distal end 16. The shaft 14 may include the sheath 30 that surrounds at least a portion of one or more lumens. The shaft 14 and sheath 30 may also include or otherwise radially surround the electrode 26 and a plurality of optical fibers 44 and 46. The sheath 30 includes an inner lumen 42 running longitudinally within the sheath 30, and the inner lumen 42 runs distally to an opening 40. The electrode 26 may be positioned (e.g., movably positioned) within the inner lumen 42 and at least a distal portion of the sheath 30. The electrode 26 may also include an electrode lumen 36.

The electrode 26 may have a “T” shape cross-section, as shown in FIG. 1. Alternatively, the electrode 26 may include various shapes. For example, the electrode 26 may include a needle-like tip. In some aspects, the electrode 26 may include specialized geometries optimized for specific medical procedures. For instance, the electrode 26 may have one or more serrated edges, for example, for cutting and/or a curved shape, for example, to help conform to the anatomy of specific tissues. Additionally, in some aspects, the electrode 26 includes an electrode lumen 36, for example, extending longitudinally through a central portion of the electrode 26.

The electrode 26 may include a widened distal portion 38. The widened distal portion 38 of the electrode 26 may be wider than an opening 40 of the sheath 30. Because of the widened distal portion 38 of the electrode 26, a portion of the electrode 26 may be positioned distally of the opening 40, for example, in both the extended and retracted configurations. Alternatively, although not shown, the electrode 26 may not include a widened portion, such that the entirety of the electrode 26 may be retracted within the sheath 30.

The electrode 26 extends from the distal end 16 and may be actuated by a conductive element or a drive wire, such that the conductive element imparts distal or proximal movement. The movable body 20 moves relative to the handle 14 to control movement of the electrode 26. For instance, the movement of the movable body 20 controls the movement of the conductive element, and the movement of the conductive element imparts proximal and distal movement to the electrode 26.

Medical device 10 includes one or more optical fibers 44, 46 that are light-emitting or light-receiving. As shown in some embodiments, the optical fibers 44, 46 may be interchangeable. The optical fibers 44, 46 may be circular or any shape corresponding to the structure of the sheath 30 and the electrode 26. Depending on the intended application, the optical fibers 44, 46 within a lumen may be positioned in various geometric arrangements. Multiple optical fibers 44, 46 can be placed at the center of the lumen, running parallel to each other. In addition, the optical fibers 44, 46 may be tapered or gradually reduced in diameter along the length of the lumen. Alternatively, a single optical fiber (e.g., 46) can be positioned at the center of the electrode lumen 36, running along its axis.

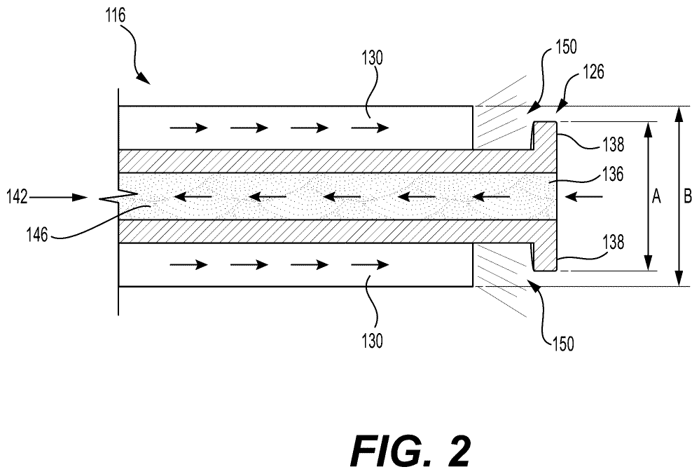

The optical fibers 44, 46 and any embodiments described below containing optical properties (sheath/lumen in FIGS. 2, 3, and 5) may also include an optical wall (not shown). The optical walls or cladding layer may include inner insulation and outer insulation. The inner insulation may help maintain optical energy within the optical fibers 44, 46 on the radially inward side. The optical walls or cladding layer may have a lower refractive index than the electrode lumen 36 comprising the optical fibers 44, 46, which causes light to be reflected and confined within the electrode lumen 36. The outer insulation may play a different role and insulate the optical fibers 44, 46 from the electrode 26 and prevent energy, in this case, electrical energy, from escaping radially inward toward the including the optical fibers 44, 46.

The optical fiber 44 may be a light-emitting optical fiber that emits light onto a target tissue. The light-emitting optical fiber 44 may extend from the handle 12 through the shaft 14 and the internal lumen of the shaft 14 or sheath 30, which may include an electrode lumen 36, to deliver light energy from the proximal end of the medical device 10 to the target site. The port 22 may be coupled to capital equipment that includes a light source. The light source and the capital equipment may then provide energy to the port 22, which may provide a light source to the optical fiber 44. The optical fiber 44 may then emit light toward a target site. The characteristics of the light source, such as its intensity, wavelength, and modulation, may be adapted to specific procedures or tissues. Alternatively, or in combination with the port 22, the handle 12 may also include one or more light sources, such as one or more diodes that may provide light energy to the optical fiber 44.

The optical fiber 46 may be a light-receiving optical fiber. The optical fiber 46 may receive light reflected from the target site, for example, from the light emitted from the optical fiber 44. The optical fiber 46 may extend from the handle 12 through the shaft 14 and the electrode lumen 36. The optical fiber 46 may then transmit the received light energy from the distal opening of the electrode lumen 36, through the components of the distal end 16, through the shaft 14 and at least to the handle 12.

In some aspects, the optical fiber 46 is coupled to the port 22 with an optical fiber connector (not shown). In this aspect, the received light energy may be proximally transmitted to capital equipment, including a processor, for example, via the port 22. Alternatively, the handle 12 may include one or more processors, control elements, or photo-diodes that may receive and process the light energy via the optical fiber 46. For example, the light-receiving optical fiber may include photo-diodes that help detect incoming light characteristics. In either embodiment with a processor, photo-diodes, a combination of both, or otherwise, the embodiments may be designed to detect light intensity, wavelength, and color.

The processor (whether in external capital equipment, included in the handle, or processed by microcontrollers) may interpret or otherwise analyze the received light profiles and output one or more instructions or signals based on the characteristics of the incoming light. For example, the light profile may match predefined characteristics of certain tissue colors, indicating that the tissue has specific attributes that require a particular response. The capital equipment's processing unit may have predefined decision logic programmed into it. This logic would include rules or algorithms that dictate what action should be taken when a light profile is detected. For example, if the detected light matches the predefined criteria (e.g., the light is red, indicating the presence of blood vessels). In that case, the processing unit may send a signal to the medical device's control system to halt the cutting action of electrode 26. Conversely, if the detected light characteristics do not match the predefined criteria for stopping (e.g., the tissue is a blue color, indicating submucosal tissue), the processing unit may allow the electrode 26 to continue its movement and cutting action.

The movement and/or energization of the electrode 26 may be stopped or prevented in one or more ways. For example, the handle 12 may include one or more warning mechanisms, such as an LED indicator, a display screen, an audible warning, a tactile warning (e.g., a vibration), etc. When the processing unit in the capital equipment detects, for example, a red-light profile from the tissue distal to or forward of the electrode 26, the processor may output a signal to the handle, and the warning mechanism may activate. The warning on the handle 12 may notify the user (e.g., a physician or technician) that a condition requiring attention has been detected.

Alternatively, in response to the signal from the capital equipment, the electrical connection between the handle 12 and the electrode 26 may be, disabled, deactivated, interrupted, or “cut off” when the processing unit in the capital equipment detects a red light. To interrupt the electrical connection, a switch or control unit may interrupt any of the electrical connections described above. The interruption or “off” option can be achieved automatically (e.g., sending a signal) or manually by a relay or switch mechanism within the handle that interrupts the connection between the electrode 26 and the hub 24 on the handle 12. In any of these aspects, the processor may send a signal that disables the energization of the electrode 26. The user may then manually activate the electrode 26 to initiate cutting by activating the relay or switch mechanism.

Capital equipment may also have a user interface through which the medical service provider, such as a physician or medical technician, can interact with the system. A user interface on the handle 12 may allow the user to decide whether to stop the electrode 26 based on the warning on the display screen. This interface might allow users to set parameters, make decisions, or override automatic actions. For example, the user may be able to override an automatic decision that halts a cutting action through the user interface.

FIG. 2 illustrates a cross-sectional view of an alternative distal end 116 of an exemplary medical device, including a sheath 130 that may form and/or serve as a light-emitting optical fiber (e.g., similar to the light-emitting optical fiber 44) and be similarly coupled to a light source, for example, via a port (similar to port 22) on the proximal most end of a handle (not shown). As discussed above with respect to sheath 30, sheath 130 includes an inner lumen 142, and the electrode 126 may be positioned within the inner lumen 142. In the embodiment, the electrode 126 may include an electrode lumen 136 including a light-receiving optical fiber 146.

The handle and its components (e.g., hub) may be similar to the handle 12 (e.g., hub 24) in FIG. 1. The light source may provide energy to the port, which may provide light to the sheath 130. The sheath 130, may then deliver light energy by extending through the handle and through the shaft 114, thereby delivering light energy from the proximal end of the medical device toward a distal end 150 of the sheath 130. The distal end 150 of the sheath 130 may emit light toward the target tissue. In an embodiment, only a portion or segmented portions of the sheath 130 may include one or more light-emitting optical fibers. For example, the sheath 130 may include an innermost portion and an outermost portion formed of a light-emitting optical fiber but include non-light emitting material positioned between an intermediate portion of the innermost and outermost portions. Alternatively, in another embodiment the innermost and outermost portions may include non-light emitting material while the intermediate portion may include light-emitting optical fiber. The sheath 130 may be configured to convey light in designated portions of the sheath as required for a specific procedure.

In embodiments, the sheath 130 may be formed of a light-conveying material. The light-conveying material may include a degree of optical transparency to prevent absorption or scattering while the sheath 130 emits light. The material may include a refractive index that closely matches that of the surrounding tissue to minimize light loss and reflection between the sheath 130 and the tissue.

The medical device also includes a light-receiving optical fiber 146 similar to the optical fiber 46, except as described herein. The optical fiber 146 may extend from a handle through a sheath 130 and an electrode lumen 136. The optical fiber 146 may receive light from a target site distal to or adjacent to the distal end 116 and electrode lumen 136. The optical fiber 146 may then transmit the received light energy from the distal end 116 through the shaft 114 and at least to the handle, which may be similarly coupled to a processor within capital equipment. For example, the capital equipment may be coupled to the port on the handle and the optical fiber 146 may extend from the port of the handle, and may be coupled to the capital equipment. However, in this embodiment, the optical fiber 146 may occupy the entirety of the electrode lumen 136 of the electrode 126 or include a larger width than the optical fiber 146. The embodiment may also include an optical wall similar to the optical wall described above (not shown). The optical wall or cladding layer may include inner insulation and outer insulation. The inner insulation may allow the optical fiber in the sheath to retain energy within the sheath 130.

In yet another embodiment (not shown), the sheath 130 receives optical or light energy rather than delivering the light energy. In this embodiment, the sheath 130 may be similarly coupled to a processor within capital equipment and act as a light-receiving optical fiber (e.g., optical fiber 46). In the embodiment, the optical fiber 146 would be the light-emitting optical fiber and be similar to optical fiber 46.

The electrode 126 is similar to electrode 26, as described above. The electrode 26 may be positioned within the inner lumen 142 and at least a distal portion of the sheath 130. The electrode 126 also includes an electrode lumen 136 and a “T” shaped widened distal portion 138. However, in the embodiment, the widened distal portion 138 may not be so wide that it inhibits light delivery by blocking the path of light emission from a distal end 150 of the sheath 130. In the embodiment, a distal end 150 of sheath 130 may be wider than the “T” shaped distal portion of the electrode 126 and include a first width “B” and a distal portion of the electrode 126 may be narrower or include a smaller second width “A.” The dimensions of the distal portion of the electrode 126 allow for intended light delivery onto a target site without being disrupted by the “T” shaped distal portion of the electrode 126.

FIG. 3 illustrates a cross-sectional view of an alternative distal end 216 of an exemplary medical device, including a handle (not shown) and a sheath 230. The handle and its components (e.g., a hub) are similar to the handle 12 (e.g., hub 24) in FIG. 1. The electrode 226 is also similar to the electrode 26 and 126 described above, except for the differences discussed herein. As discussed above with respect to sheath 30, sheath 230 includes an inner lumen 242, and the electrode 226 may be positioned within the inner lumen 242. The electrode 226 may include an electrode lumen 236 including a light-receiving optical fiber 246.

The distal end 216 includes a sheath 230, similar to the sheath 130. The sheath 230 and the optical fiber 246 are configured to emit and receive light, respectively. The sheath 230 may emit light by extending through the handle and through the shaft thereby delivering light energy from the proximal end of the medical device toward a distal end 250 of the sheath 230. As shown, the distal end 250 of the sheath 230 may emit light toward the target tissue. Similar to FIG. 2, the distal end 250 of the sheath 230 may be wider than the “T” shaped distal portion of the electrode 226. The widened distal portion 238 may not be so wide that it inhibits light delivery by blocking the path of light emission from a distal end 250 of the sheath 230. For example, the sheath 230 may be wider or include a first width “D,” while a distal portion of the electrode 226 may be narrower or include a second width “C.” As discussed above, the dimensions of the distal portion of the electrode 226 allow for intended light delivery onto a target site without being disrupted by the “T” shaped distal portion of the electrode 226.

The optical fiber 246 is similar to optical fiber 146, except the dimensions of optical fiber 246, may be adjusted to form a fluid delivery portion 260, for example, within a portion of the electrode lumen 236. The optical fiber 246 may have a smaller width to allow space for the fluid delivery portion 260. The fluid delivery portion 260 may be used for fluid delivery or fluid injection. Injecting fluid into the submucosal layer beneath the examined or treated tissue can create a cushion or lift that may separate the mucosal layer from underlying structures.

A fluid delivery portion 260 may occupy a space adjacent to the optical fiber 246. In this embodiment, the optical fiber 246 may occupy more space within electrode lumen 236 than the fluid delivery portion 260. However, the optical fiber 246 and the fluid delivery portion 260 may include various dimensions depending on the requisite function. For example, in an alternative embodiment, the fluid delivery portion 260 may have a larger width than the optical fiber 246 and occupy more space than the optical fiber 246 within the electrode lumen 236 of electrode 226. The varying dimensions of electrode lumen 236, the optical fiber 246, and the fluid delivery portion 260 may help increase or decrease the pressure of the fluid delivered through the fluid delivery portion 260.

The fluid delivery portion 260 may receive fluid, initially, by the handle coupled to a fluid source (not shown) via a “fluid port” (not shown) to allow for a proximal fluid connection. The lumen of the fluid delivery portion 260 or the fluid delivery portion 260 may be configured to be in fluid communication with the fluid port via a support lumen that connects to the fluid port (not shown) and through a proximal support (not shown) that supports the connection between the internal lumen and the support lumen in the fluid port. The fluid port may be positioned on a proximal portion of a main body, for example, a proximal end of the main body. Alternatively, the fluid port may be positioned on a distal or central portion of the main body. Regardless of the position of the fluid port, the fluid port may include a one-way valve, a seal, threading, and/or any appropriate element to help maintain a secure connection between the handle and the fluid source, minimize or prevent back-flow (e.g., fluid flowing proximally out of port), and/or minimize or prevent leakage. In one example, a one-way valve may include an outer housing containing an inner elastomeric and/or gelatinous sealing member (not shown). In an embodiment, the port may be configured to receive both optical energy and fluid, acting as a fluid port. The embodiment would include the embodiments described for the fluid port and similar aspects of the port 22.

The fluid delivery portion 260 may extend longitudinally through the main body of the handle and shaft 214 to fluidly connect the fluid proximally received by the fluid port to the electrode 226. The fluid delivered distally by the fluid port may surround at least a portion of a conductive element in the main body. In one aspect, the conductive element may include one or more layers of insulation to help insulate the conductive element from fluid in the internal lumen.

The fluid may be delivered distally through the electrode lumen 236 in at least two ways. In one embodiment, the fluid delivery portion 260 may itself form a channel to deliver fluid from an opening of the electrode lumen 236. Fluid may be delivered by the fluid delivery portion 260 by the lumen within the fluid delivery portion 260, which may serve as an internal pathway through which fluids are transported from a proximal end and delivered through the opening of the electrode lumen 236. The fluid delivery portion 260 may run longitudinally and have a consistent diameter or vary in size along its length.

In another embodiment, the fluid delivery portion 260 may be in fluid communication with an insulation tip lumen (not shown) to form a channel to deliver fluid from the opening of the electrode lumen 236. In one aspect, the insulation tip surrounds or covers the opening of the electrode lumen 236. The insulation tip in combination with the fluid delivery portion 260 may form a fluid channel that extends through both the fluid delivery portion 260 and insulation tip to deliver (e.g., inject) fluid to a target site (e.g., within or between layers of tissue to raise, separate, flush, or otherwise treat tissue).

Moreover, the insulation tip may abut tissue, and electrode 226 may be energized while the insulation tip helps to insulate the tissue that the insulation tip abuts against. The electrode 226 may be energized, and the exposed portion of electrode 226 (e.g., T-shaped portion) may be used to cut, dissect, ablate, mark, coagulate, cauterize, or otherwise treat tissue. Additionally, approximately one half of the insulation tip may extend distally beyond the distal tip, which may help insulate tissue abutting the distal end 216 when the electrode 226 is energized.

FIG. 4 illustrates a cross-sectional view of a further alternative embodiment of a distal end 316, including a sheath 330 and an electrode 326. The sheath 330 may be coupled to a handle, as discussed above. The electrode 326 is similar to the electrode 26, described above (FIG. 1). As discussed above with respect to sheath 30, sheath 330 includes an inner lumen 342, and the electrode 326 may be positioned within the inner lumen 342. The electrode 326 may include an electrode lumen 336 including a light-emitting optical fiber 344 and a light-receiving optical fiber 346. The embodiment of FIG. 4 includes at least a sheath 330, including at least a lumen 331 between an inner wall 370A and an outer wall 370B. The sheath 330 may include at least one lumen 331 for fluid delivery, fluid injection, or suction. The description of the fluid delivery portion 260 (FIG. 3) may apply to the lumen 331 of sheath 330. However, in this embodiment, the lumen 331 of the sheath 330 is configured to deliver fluid.

The lumen (“lumen”) 331 of sheath 330 may receive fluid in the same manner as the fluid delivery portion 260. The lumen 331 of sheath 330 may extend through or otherwise be fluidly connected to the handle and through one or more portions of sheath 330. The sheath 330 may receive fluid, initially, by the handle, which may be coupled to a fluid source (not shown) via a fluid port to allow for a fluid connection. In some aspects, the sheath 330 may include an inner wall 370A and outer wall 370B. The inner wall 370A and outer wall 370B may help form lumen 331 of sheath 330. Lumen 331 may help deliver fluid distally, for example, around one or more portions of electrode 326. The walls may also create a barrier that can isolate the fluid within the sheath 330 from the surrounding tissues or structures.

The lumen 331 of sheath 330 may deliver fluid similarly to the fluid delivery portion 260, except that fluid may be delivered from the distal end 350 of sheath 330. In one embodiment, the lumen 331 of sheath 330 may itself form a channel to deliver fluid from the distal end 350 of sheath 330. The distal end 350 may include at least a distal opening 340. The distal opening 340 of the one or more fluid delivery or suction application lumens may be at least partially radially outside of a distal end of the electrode 326. In another embodiment, the lumen 331 of sheath 330 may be in fluid communication with the one or more lumens with an insulation tip or end cap (not shown) to form one or more channels to deliver fluid from the distal end 350 or distal opening 340 of the lumen 331 of the sheath 330. In either embodiment, the widened distal portion 338 of the electrode 326 may not be so wide that it inhibits fluid delivery by blocking the path of fluid delivery from the distal end 350 of sheath 330. Therefore, a distal end 350 of the sheath 330 or the radially outer edge(s) of lumen 331 may be wider or include a first width “F” and a distal portion of the electrode 326 may be narrower or include a second width “E.”

In another embodiment, the lumen 331 of sheath 330 may be a suction lumen. The lumen 331 may apply negative pressure, for example, to help to aspirate unwanted substances. The suction lumen may be configured similarly as the fluid delivery lumen, except that the proximal suction port may be connected to an external suction source or system rather than, or in addition to, a fluid port. The external suction source may be, for example, a vacuum pump, a dedicated suction machine, or a central vacuum system. The sheath 330 may include a plurality of lumens, for example, including both one or more suction lumens and one or more fluid delivery lumens. Alternatively or additionally, the sheath 330 may also include one or more lumens (e.g., the lumen 331) that can alternate to deliver fluid and perform suction, as needed during a procedure.

The presence of separate lumens may allow for independent channels of fluid delivery. A plurality of lumens may have distinct functions and/or be used for different types of fluids, depending on the requirements of the medical procedure. The presence of multiple lumens may provide flexibility in fluid management during procedures, allowing for the simultaneous delivery of various fluids or the controlled delivery of a single fluid through different channels. For example, one lumen within sheath 330 may be dedicated to a saline solution for tissue irrigation, while another lumen may deliver a contrast agent for imaging enhancement, while yet another lumen is used for suction.

The sheath 330 includes an inner lumen 342 positioned with the electrode 326 that may include the electrode lumen 336. The light emitting optical fiber 344 and the light-receiving optical fiber 346 may be positioned within the electrode lumen 336. The optical fibers 344, 346 are similar to the optical fibers 44 and 46 in FIG. 1. For example, a light-emitting optical fiber 344 may be coupled (e.g., directly or indirectly) to a light source (not shown). The optical fiber 344 may then emit light to the target site. Additionally, a light-receiving optical fiber 346 may receive light energy from the target site. The optical fiber 346 may transmit received light energy from the distal end 316 through the shaft and at least through a handle. The received light energy may be proximally transmitted to capital equipment, including a processor, for example, via a port. The processor (whether in external capital equipment or included in the handle) may interpret the received light or light profiles and make decisions to turn the electrode on or off based on the characteristics of the incoming light or light profiles.

FIG. 5 illustrates a cross-sectional view of another alternative embodiment of the distal end 416, including a sheath 430 and an electrode 426. The embodiment combines various features of FIGS. 2-4. As discussed above with respect to sheath 30, sheath 430 includes an inner lumen 442, and the electrode 426 may be positioned within the inner lumen 442. In the embodiment, the electrode 426 may include an electrode lumen 436 including a light-receiving optical fiber 446. The light-receiving optical fiber 446 may be similar to light-receiving optical fiber 146, described above. The sheath 430 may also include at least two lumens (431, 432) between an inner wall 470A and an outer wall 470B. For example, sheath 430 may include a lumen 432 that may receive an optical fiber 444 to transmit or emit light to the tissue, and a lumen 431 that may be used for fluid delivery. The description of the sheath 130 may apply to the lumen 432 of sheath 430, and the description of the lumen 331 of sheath 330 may apply to the lumen 431 of sheath 430.

Similar to optical fiber 246 in FIG. 3, the optical fiber 446 may transmit the received light energy from an opening of the electrode lumen 436 through the shaft and at least to the handle. The received light energy may then be proximally transmitted to a processor that interprets the received light profiles and make decisions to turn the electrode on or off based on the characteristics of the incoming light, as described above.

The sheath 430 includes at least two lumens 432 and 431 that may include light emission or fluid delivery. The sheath 430 may include an inner wall 470A and outer wall 470B. The inner wall 470A and outer wall 470B may form the lumens (e.g., the fluid lumen 431 and the light-emitting lumen 432) that may act as a passage for fluid, suction, or light delivery. The fluid, suction, or light delivery may be delivered through a distal opening 440 that may be at least partially radially outside of a distal end of the electrode 426. In the embodiment of FIG. 5, the lumen 432 may act as a light-emitting optical fiber and include a light-emitting optical fiber 444 to transmit light (e.g., similar to the sheath 130). To emit light, a light source may provide energy to a port (e.g., similar to port 122), and then to the light-emitting optical fiber 444 within the sheath 430 and lumen 432. In some aspects, instead of emitting light energy, the optical fiber 444 within the lumen 432 receives light energy to be proximally transmitted (see, e.g., optical fiber 46) to capital equipment, including a processor, for example, via port.

The sheath 430 and lumen 431 may be in fluid communication with a fluid source. The lumen 431 of sheath 430 may be similar to the lumen 331 of sheath 330. The lumen 431 of sheath 430 may receive one or more fluids in a similar manner as the fluid delivery portion 260 or the lumen 331 in the sheath 330. As discussed above, the widened distal portion 438 may not be so wide that it inhibits fluid delivery by blocking the path of fluid delivery from the distal end 450 of sheath 430. Therefore, a distal end 450 of the sheath 430 or the radially outer edge(s) of lumen 432 and 431 may be wider and include a first width “H,” and a distal portion of the electrode 426 may be narrower and include a second width “G.”

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
an insertion portion, including a shaft that includes a sheath and an electrode, wherein the sheath includes a sheath lumen, wherein the electrode is positioned within at least a distal portion of the sheath, wherein the electrode includes an electrode lumen, and wherein the electrode is configured to deliver energy to a target site;
a light-emitting optical fiber configured to deliver light to the target site, wherein at least a portion of the light-emitting optical fiber is positioned within the sheath or the electrode lumen; and
a light-receiving optical fiber configured to receive light from the target site, wherein at least a portion of the light-receiving optical fiber is positioned within the electrode lumen.

2. The medical device of claim 1, wherein the electrode has a “T” shaped cross-section with an electrode shaft and a widened distal portion, wherein a portion of the electrode extends distally of the sheath lumen, and wherein the widened distal portion is wider than an opening of the sheath lumen.

3. The medical device of claim 1, wherein at least a portion of the sheath is configured to convey light and form the light-emitting optical fiber.

4. The medical device of claim 3, wherein a distal end of the sheath includes a first width, wherein a distal end of the electrode includes a second width, and wherein the second width is less than the first width.

5. The medical device of claim 1, wherein a width of the electrode lumen is greater than a width of the light-receiving optical fiber, such that a portion of the electrode lumen forms a fluid delivery portion that occupies a space adjacent to the light-receiving optical fiber.

6. The medical device of claim 1, wherein the light-emitting optical fiber is positioned within the electrode lumen.

7. The medical device of claim 6, wherein the sheath includes one or more fluid delivery or suction application lumens.

8. The medical device of claim 7, wherein the one or more fluid delivery or suction application lumens includes at least two fluid delivery or suction application lumens.

9. The medical device of claim 7, wherein respective distal openings of the one or more fluid delivery or suction application lumens are at least partially radially outside of a distal end of the electrode.

10. The medical device of claim 7, wherein the sheath includes the light-emitting optical fiber and the one or more fluid delivery or suction application lumens.

11. The medical device of claim 1, wherein the medical device includes a handle, wherein the light-emitting optical fiber extends from the handle through a portion of the shaft to deliver light to the target site.

12. The medical device of claim 11, wherein the handle of the medical device includes a port, wherein the port is coupled to a light source that provides light to the light-emitting optical fiber.

13. The medical device of claim 11, wherein the light-receiving optical fiber extends through the electrode lumen, through a shaft lumen, to the handle, and to a processor, wherein the processor analyzes light received by the light-receiving optical fiber to generate a light profile.

14. The medical device of claim 13, wherein based on the generated light profile, the processor is configured to deactivate the electrode.

15. The medical device of claim 14, wherein when the processor detects a red light the processor sends a signal to disable energization of the electrode.

16. A medical device, comprising:
a handle including a port and a hub;
an insertion portion, including a shaft that includes a sheath and an electrode, wherein the sheath includes a sheath lumen, wherein the electrode is positioned within at least a distal portion of the sheath, wherein the electrode includes an electrode lumen, and wherein the electrode is configured to deliver energy to a target site;
a light-emitting optical fiber configured to deliver light to the target site, wherein at least a portion of the light-emitting optical fiber is positioned within the sheath or the electrode lumen; and
a light-receiving optical fiber configured to receive light from the target site, wherein at least a portion of the light-receiving optical fiber is positioned within the electrode lumen;
wherein at least a portion of the sheath directs optical energy distally.

17. The medical device of claim 16, wherein the sheath is configured to emit light, wherein the sheath emits light toward the target site, wherein at least a portion of the sheath is configured to convey light and form the light-emitting optical fiber.

18. The medical device of claim 16, wherein a width of the electrode lumen is greater than a width of the light-receiving optical fiber, such that a portion of the electrode lumen forms a fluid delivery portion that occupies a space adjacent to the light receiving optical fiber.

19. The medical device of claim 17, wherein the light-receiving optical fiber is coupled to a processor;
wherein the processor analyzes light received by the light-receiving optical fiber to generate a light profile, wherein, based on the generated light profile, the processor is configured to deactivate the electrode; wherein when the processor detects a red light, the processor sends a signal to disable energization of the electrode.

20. A medical device, comprising:
an insertion portion, including a sheath and an electrode;
wherein the sheath includes a sheath lumen, wherein the sheath includes one or more fluid delivery or suction application lumens;
wherein the electrode is positioned within at least a distal portion of the sheath lumen, wherein the electrode includes an electrode lumen, and wherein the electrode is configured to deliver energy to a target site;
a light-emitting optical fiber configured to deliver light to the target site, wherein at least a portion of the light-emitting optical fiber is positioned within the sheath or the electrode lumen; and
a light-receiving optical fiber configured to receive light from the target site, wherein at least a portion of the light-receiving optical fiber is positioned within the electrode lumen;
wherein the light-receiving optical fiber is coupled to a processor, wherein the processor analyzes light received by the light-receiving optical fiber to generate a light profile, wherein based on the generated light profile, the processor is configured to deactivate the electrode.
